(19) United States
(12) Patent Application Publication
(10) Pub. No.: US 2006/0097189 A1
(43) Pub. Date: May 11, 2006

(54) Disinfecting system for a toilet

(75) Inventor: Seng Ming Lim, Norman, OK (US)

Correspondence Address: CARLSON, GASKEY & OLDS, P.C., 400 WEST MAPLE ROAD, SUITE 350, BIRMINGHAM, MI, 48009, US

(73) Assignee: Masco Corporation of Indiana

(21) Appl. No.: 10/983,524

(22) Filed: Nov. 8, 2004

(51) Int. Cl. A61L 2/10

(52) U.S. Cl. 250/492.1; 250/504 R

(57) ABSTRACT

The present invention provides a disinfecting system that uses ultraviolet light to disinfect a toilet seat and toilet bowl. The disinfecting system is mounted to a toilet seat cover. A control is used to activate the system when the toilet seat cover is lowered. An indicator light can be mounted to an opposing side of the toilet seat. The indicator light can be used to provide an indication that the system is currently operating or to indicate other status changes in the system.

### BACKGROUND OF THE INVENTION

The present invention relates to an apparatus and method for disinfecting toilet seats and bowls. More particularly, the invention uses ultraviolet light to disinfect a toilet seat and bowl.

Toilet seats and bowls require disinfecting to prevent the spread of infection and to maintain a clean bathroom environment. The most common method of sanitizing toilets is manually. However, frequent disinfecting is needed to maintain a sanitized toilet. Devices have been invented to reduce the frequency of required disinfecting and maintain a cleaner toilet. For example, paper coverings for the toilet seats are commonly used to provide a sanitized surface. However, this method is inconvenient for home use.

Automated disinfecting systems have also been invented to reduce the efforts of the user. Known automated disinfecting systems require complicated mechanisms. These mechanisms are expensive to purchase and maintain. For example, a water or spraying device has been used to disinfect the toilet bowl or seat. Additional plumbing is required to operate the sprayer. In addition, the known systems have sprayers which target disinfecting either a toilet bowl or a toilet seat, not both.

One known system uses an ultraviolet light mounted within a toilet bowl. The light is directed upward, away from the bowl. However, this system is used to disinfect the toilet user and does not disinfect the toilet itself.

Thus, a system that disinfects both a toilet seat and toilet bowl without complicated and expensive equipment is needed.

### SUMMARY OF THE INVENTION

The present invention provides a disinfecting system that uses ultraviolet light, such as a light source, for disinfecting a toilet seat and toilet bowl. The disinfecting system is mounted to a toilet seat cover, or another area where it can be directed toward the toilet seat or toilet bowl. A control is used to activate the system when the toilet seat cover is lowered. The disinfecting system runs for a predetermined amount of time required for disinfecting the toilet seat and bowl. The system is automatically turned off after the predetermined time. In a disclosed embodiment, when someone lifts the toilet seat during operation of the system, the control would send a signal to turn off the UV light. Thus, the system does not prevent use of the toilet.

Additionally, an indicator light can be mounted to an opposing side of the toilet seat. When the toilet seat is lowered, the indicator light can be easily seen. The indicator light can be used to alert a user the disinfecting system is currently operating, or other status changes in the system.

### BRIEF DESCRIPTION OF THE DRAWINGS

The various features and advantages of this invention will become apparent to those skilled in the art from the following detailed description of the currently preferred embodiment. The drawings that accompany the detailed description can be briefly described as follows:

FIG. 1 is a general view of a toilet and toilet seat cover with the disinfecting system of the present invention; and

FIG. 2 is an exploded view of system configuration.

### DETAILED DESCRIPTION OF THE PREFERRED EMBODIMENT

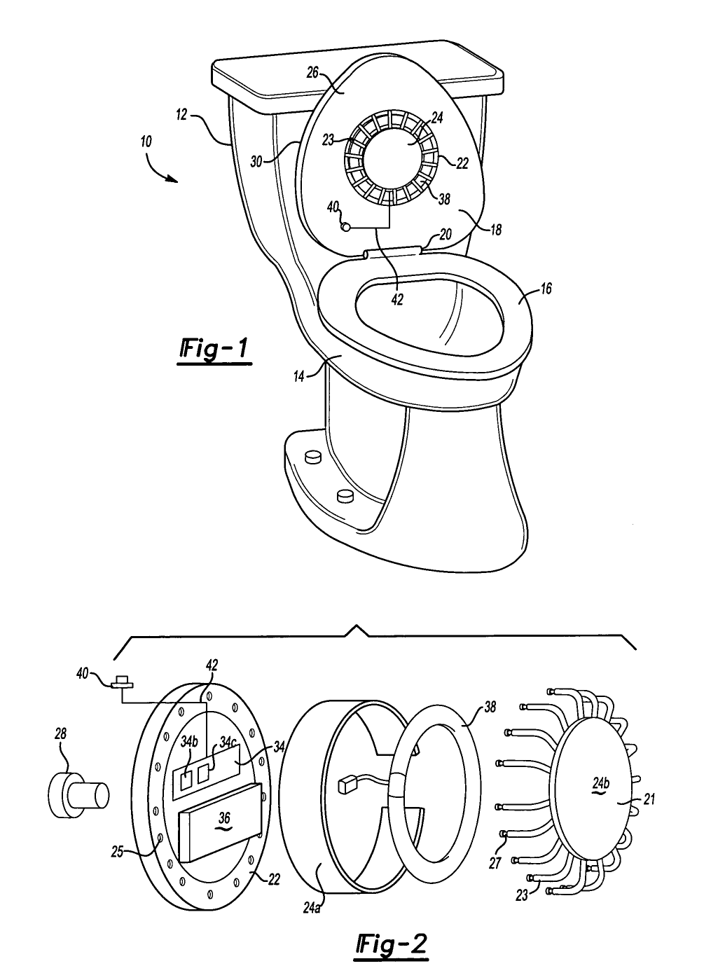

FIG. 1 illustrates a general view of an example disinfecting system 10 of the present invention. A toilet 12 has a toilet bowl 14. A toilet seat 16 and seat cover 18 are located on top of the toilet bowl 14. A hinge 20 allows the toilet seat 16 and seat cover 18 to be raised and lowered as needed.

The disinfecting system 10 includes a housing 22 and a housing cover 24. In one embodiment, the housing 22 is mounted on a first side of the seat cover 18. The housing 22 is mounted by known conventional means. For example, in one embodiment, an indicator 28 (Shown in FIG. 2) is mounted on an opposing side 30 of the seat cover 18. A through hole in the seat cover 18 allows wiring to connect the indicator 28 and the housing 22. The indicator 28 may also act as a fastener in retaining the housing 22 to the seat cover 18. The housing 22, and housing cover 24 may be made from stainless steel or other materials that are easily disinfected. The housing 22 may be mounted in other locations on the toilet that allow the disinfecting system 10 to be directed toward the toilet bowl 14 or the toilet seat 16.

FIG. 2 illustrates an exploded view of one embodiment of the disinfecting system 10. Within the housing 22 are a control 34, a rectifier 36, and a UV light 38. The rectifier 36 is connected to the control 34 to provide power to the disinfecting system. The control 34 may be a switch in one example. Further, in this embodiment, the housing cover 24 is formed of two parts. A first cover part 24a surrounds the internal components. A second cover part 24b has a plate 21 to conceal the control 34 and rectifier 36. Ribs 23 extend from the plate 21 for attaching the second cover part 24b to the housing 22. Spaces between the ribs 23 allow the UV light 38 to be exposed. The ribs 23 fit into slots 25 in the housing 22. Indentations 27 on the ribs 23 hold the ribs 23 within the slots to retain the cover 24 to the housing 22. However, the ribs can be removed from the slots 25 to remove the cover 24. The cover 24a and 24b is removably attached to the housing 22 to allow the UV light 38 to be replaced when necessary. Other sources of electromagnetic radiation can be used to disinfect as an alternative to the UV light 38.

In one embodiment, the control 34 includes a switch 40. The switch 40 is connected to the control 34 by wiring 42. The switch 40 can be placed in any location that will result in activation of the UV light 38 when the seat cover 18 is lowered. When the seat cover 18 is lowered to the seat 16, the switch 40 sends a signal to the control 34. The control 34 turns the UV light 38 on. After a predetermined amount of time has passed, the control 34 will automatically shut-off the UV light 38. In addition, if the seat cover 18 is lifted while the UV light 38 is on the switch 40 signals the control 34. The control 34 will then shut off the UV light 38. The control 34 includes a timer 34b and a look-up table 34c to record the predetermined amount of time and to track the amount of time that has passed.

The indicator 28 is connected to the control 34 as well. The indicator 28 will turn on when the UV light 38 is on, to provide an indication that the system is operating. In one embodiment, the indicator 28 is a light. The indicator 28 may blink or flash to indicate a change in status, electronics out of order, lamp failure, etc. Alternately, the indicator 28 is a device which provides audible indication by emitting a periodic beeping sound to indicate the system status, or to indicate a change in the status. In the place of the switch 40, control 34 can alternately include a timing device which can be set to activate the disinfecting system 10 at a predetermine time, or in predetermined intervals.

In an alternate embodiment the rectifier 36 is replaced by a battery and battery mount. In this case the indicator 28 may provide an additional signal to alert to a low battery status. For example, the additional signal for a visual indicator 28 could include flashing the light, or for an audible indicator 28 a change in tone.

Although a preferred embodiment of this invention has been disclosed, a worker of ordinary skill in this art would recognize that certain modifications to the disclosed example would come within the scope of this invention. For that reason, the following claims should be studied to determine the true scope and content of this invention.

1. A toilet disinfecting system comprising:
a light mountable to a toilet to be directed toward a toilet seat and a toilet bowl; and
a control for activating said light.

2. (canceled)

3. The toilet disinfecting system of claim 1, wherein said light is powered by at least one battery.

4. The toilet disinfecting system of claim 1, wherein said control includes a switch attached to a toilet seat cover.

5. The toilet disinfecting system of claim 4, wherein said switch is attached to said toilet seat cover in a location to be activated when said toilet seat cover is closed.

6. The toilet disinfecting system of claim 4, wherein said light is shut off when said toilet seat cover is in an open position.

7. The toilet disinfecting system of claim 1, wherein said control includes a timer.

8. The toilet disinfecting system of claim 1, wherein said light is removably mounted to said toilet seat cover.

9. The toilet disinfecting system of claim 1, wherein said light is an ultraviolet light.

10. A toilet comprising:
a disinfecting system mountable to a first side of a toilet seat cover, including an ultraviolet light mountable to said first side of said toilet seat cover and directed toward a toilet seat and toilet bowl when said toilet seat cover is closed;
a control mountable to said toilet seat cover and connected to said ultraviolet light for activating said ultraviolet light when said toilet seat cover is closed.

11. The toilet disinfecting system of claim 10, wherein said ultraviolet light is powered by at least one battery.

12. The toilet disinfecting system of claim 10, wherein said control includes a timer.

13. The toilet disinfecting system of claim 10, wherein said control includes a switch.

14. The toilet disinfecting system of claim 10, wherein said control includes a sensor for sensing a position of the toilet seat cover.

15. A method of disinfecting a toilet seat comprising:
a) providing a toilet with a toilet seat and toilet seat cover;
b) activating a light when the toilet seat cover is closed to direct the light at the toilet seat and a toilet seat bowl.

16. The method of claim 15, including;
c) shutting off the light after a predetermined amount of time has passed.

17. The method of claim 15, including:
d) shutting off the light if the toilet seat cover is opened during step a).

18. The method of claim 15, wherein step a) includes activating a switch to activate the light when the toilet seat cover is closed.

19. The method of claim 18, wherein step a) includes depressing said switch when the toiler seat cover is closed.
